(19) United States
(12) Patent Application Publication
(10) Pub. No.: US 2008/0221521 A1
(43) Pub. Date: Sep. 11, 2008

(54) Methods and Systems For Detecting an Occlusion

(76) Inventors: Steven P. Getz, Malvern, PA (US); Ian Maxwell Shipway, Ardmore, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/584,325

(22) PCT Filed: Dec. 28, 2004
(86) PCT No.: PCT/US04/41407

§ 371 Date: Oct. 12, 2007

Related U.S. Application Data
(60) Provisional application No. 60/533,004, filed on Dec. 29, 2003.

(51) Int. Cl. A61M 5/14

(52) U.S. Cl. 604/151

(57) ABSTRACT

Systems and methods for detecting an occlusion may include receiving a signal corresponding to a first force needed to deliver a first material through the tube. Furthermore, the systems and methods may include indicating that an occlusion exists if the first force is greater than a baseline value plus a delta value, the baseline value being assigned a value equal to the force necessary to deliver the first material through the tube in an un-occluded state and the delta value being assigned a value configured to create a desired level of sensitivity.

### RELATED APPLICATION

Under provisions of 35 U.S.C. §119(e), this Application claims the benefit of U.S. Provisional Application No. 60/533,004, filed Dec. 29, 2003, which is incorporated herein by reference.

### BACKGROUND OF THE INVENTION.

I. Field of the Invention

The present invention generally relates to detecting an occlusion. More particularly, the present invention relates to detecting an occlusion, and even more particularly, for example, to detecting an occlusion in an ambulatory infusion pump.

II. Background Information

Devices, such as ambulatory infusion pumps, may deliver material, such as insulin, through a tube and hollow needle (the infusion set) into a user's body. At times the infusion set may become blocked or “occluded”. This situation may result in the user not receiving one or more full doses of insulin. Because it is medically dangerous for a patient not to receive a full dose of medication, this situation needs to be detected and the user needs to be warned when this situation occurs.

With an insulin infusion pump, for example, the force required to deliver the insulin through the infusion set increases when an occlusion is present in the system. Conventional occlusion detection methods look for the force to rise above a predetermined level, or to rise above a predetermined delta added to an initial delivery force. These methods suffer from either not detecting the occlusion early enough or, because they are too sensitive, provide false alarms due to long slow force variations unrelated to an occlusion.

### SUMMARY OF THE INVENTION

Consistent with embodiments of the present invention, systems and methods are disclosed for detecting an occlusion.

In accordance with an embodiment of the invention, a method for detecting an occlusion comprises measuring a first force needed to deliver a first material through the tube, determining that an occlusion exists in the tube if the first force is greater than a baseline value plus a delta value, the baseline value being assigned a value equal to the force necessary to deliver the first material through the tube in an un-occluded state, and the delta value being assigned a value selected to create a desired level of sensitivity if the first force is less than or equal to the baseline value plus the delta value, and in the event a turbulence factor is less than a threshold value, the baseline value is equal to a second force, the second force being a low-pass filtered version of the first force, the turbulence factor being a low-pass filtered version of the absolute value of the difference between the first force and the second force, measuring a third force needed to deliver a second material through the tube, and determining that an occlusion exists in the tube if the third force is greater than the baseline value plus the delta value.

According to another embodiment of the invention, a system for detecting an occlusion comprises a memory storage for maintaining a plurality of data registers, and a processing unit coupled to the memory storage, wherein the processing unit is operative to receive a first force needed to deliver a first material through the tube, and determine that an occlusion exists in the tube if the first force is greater than a baseline value plus a delta value, the baseline value being assigned a value equal to the force necessary to deliver the first material through the tube in un-occluded state, and the delta value being assigned a value selected to create a desired level of sensitivity. If a turbulence factor is less than a threshold value, the baseline value is equal to a second force, being a low-pass filtered version of the first force, the turbulence factor being a low-pass filtered version of the absolute value of the difference between the first force and the second force, measure a third force needed to deliver a second material through the tube, and determine that an occlusion exists in the tube if the third force is greater than the baseline value plus the delta value.

According to another embodiment of the invention, a computer-readable medium which stores a set of instructions which when executed performs a method for detecting an occlusion, the method executed by the set of instructions comprising measuring a first force needed to deliver a first material through the tube, determining that an occlusion exists in the tube if the first force is greater than a baseline value plus a delta value, the baseline value being assigned a value equal to the force necessary to deliver the first material through the tube in an un-occluded state, and the delta value being assigned a value selected to create a desired level of sensitivity if the first force is less than or equal to the baseline value plus the delta value, and in the event a turbulence factor is less than a threshold value, the baseline value is equal to a second force, the second force being a low-pass filtered version of the first force, the turbulence factor being a low-pass filtered version of the absolute value of the difference between the first force and the second force, measuring a third force needed to deliver a second material through the tube, and determining that an occlusion exists in the tube if the third force is greater than the baseline value plus the delta value.

It is to be understood that both the foregoing general description and the following detailed description are exemplary and explanatory only, and should not be considered restrictive of the scope of the invention, as described and claimed. Further, features and/or variations may be provided in addition to those set forth herein. For example, embodiments of the invention may be directed to various combinations and sub-combinations of the features described in the detailed description.

### BRIEF DESCRIPTION OF THE DRAWINGS

The accompanying drawings provide a further understanding of the invention and, together with the detailed description, explain features and embodiments of the invention. In the drawings:

FIG. 1 is a block diagram of an exemplary occlusion detection system, consistent with an embodiment of the present invention; and

FIG. 2 is a flow chart of an exemplary method for detecting an occlusion consistent with an embodiment of the present invention.

### DETAILED DESCRIPTION

The following detailed description refers to the accompanying drawings. Wherever possible, the same reference numbers are used in the drawings and the following description to refer to the same or similar parts. While several exemplary embodiments and features of the invention are described herein, modifications, adaptations and other implementations are possible, without departing from the spirit and scope of the invention. For example, substitutions, additions or modifications may be made to the components illustrated in the drawings, and the exemplary methods described herein may be modified by substituting, reordering or adding steps to the disclosed methods. Accordingly, the following detailed description does not limit the invention. Instead, the proper scope of the invention is defined by the appended claims.

Instead of having the conventional system's fixed baseline value, the baseline value may vary in embodiments consistent with the invention. How and when the baseline value varies may determine, for example, the sensitivity to small deliveries (of insulin or other medicines, for example), and prevent slow changing outside force variations from causing false occlusion warnings. Several variables may be used in embodiments consistent with the invention. For example, these variables may include, but are not limited to:

i) F may comprise the force used to deliver material (insulin, for example). This can be either pre- or post-delivery;

ii) “Filtered-F” may comprise a low-pass filtered version of the force F;

iii) “Baseline” may comprise a value at which the un-occluded force F should stay near;

iv) “Delta” may comprise the amount of force above the baseline value that may trigger an occlusion warning. This is generally set to one of several fixed values which determine the level of sensitivity desired; and

v) “Turbulence” may comprise a measure of how smooth F is relative to “Filtered-F”. A larger number indicates that F is wandering further away from “Filtered-F”.

An algorithm for detecting an occlusion may include recalculating at least some or all of the above variables each time a delivery is made. First, F may be checked to make sure it does not exceed the baseline value plus the delta. If F does exceed the baseline value plus the delta, then an occlusion condition may exist. If no occlusion exists (for example, when F is less than or equal to the baseline value plus the delta), other variables may be recalculated and updated. This algorithm may be repeated for each delivery.

Turbulence may be a function of the volatility of F relative to filtered-F. For example, one representation of turbulence may be a low-pass filtered version of the absolute value of the difference between F and filtered-F. If the turbulence is less than a predefined level, then a new baseline value may be established by setting the baseline value equal to the filtered-F.

Reading the force before the delivery may allow forces not related to the occlusion to relax, and therefore, to not interfere with true occlusion detection. A large delivery could be used to desensitize the algorithm for a short period. This may allow for a combination of deliveries of large “boluses” mixed with smaller “basal” deliveries. If the previous delivery was too recent, or very large, then a short term desensitizing of the algorithm may be advantageous. This may facilitate multiple deliveries back-to-back such as extended or combined deliveries that occur immediately before or after a basal delivery.

For example, the algorithm may be desensitized for a period after a new cartridge of insulin is loaded into an insulin pump. This may be advantageous because there may be a period after a new cartridge is loaded in which delivery force will vary more. Furthermore, the algorithm could be dynamic depending on the size of the delivery. For example, the delta could be a function of the number of units delivered in the last n minutes.

Moreover, algorithm results (for example, values of the aforementioned variables) could be saved in a memory for later analysis in order to refine some or all of the variables used by the algorithm. For example, the parameters could be stored in a non-volatile memory that may be read or altered by, for example, a manufacturer through an interface port. This may allow custom variations of the algorithm that may help tailor the device to the needs of a particular user.

Consistent with an embodiment of the invention, a system for detecting an occlusion may comprise a memory storage for maintaining a plurality of data registers and a processing unit coupled to the memory storage. The processing unit may be operative to receive a first force needed to deliver a first material through the tube. Furthermore, the processing unit may be operative to indicate that an occlusion exists in the tube if the first force is greater than a baseline value plus a delta value, the baseline value being assigned a value equal to the force necessary to deliver the first material through the tube in an un-occluded state and the delta value being assigned a value selected to create a desired level of sensitivity. Moreover, the processing unit may be operative to set, if the first force is less than or equal to the first baseline plus the delta value, and if a turbulence factor is less than a threshold value, the baseline value equal to a second force, this second force being a low-pass filtered version of the first force, the turbulence factor being a low-pass filtered version of the absolute value of the difference between the first force and the second force. In addition, the processing unit may be operative to receive a third force needed to deliver a second material through the tube and indicate that an occlusion exists in the tube if the third force is greater than the baseline plus the delta value.

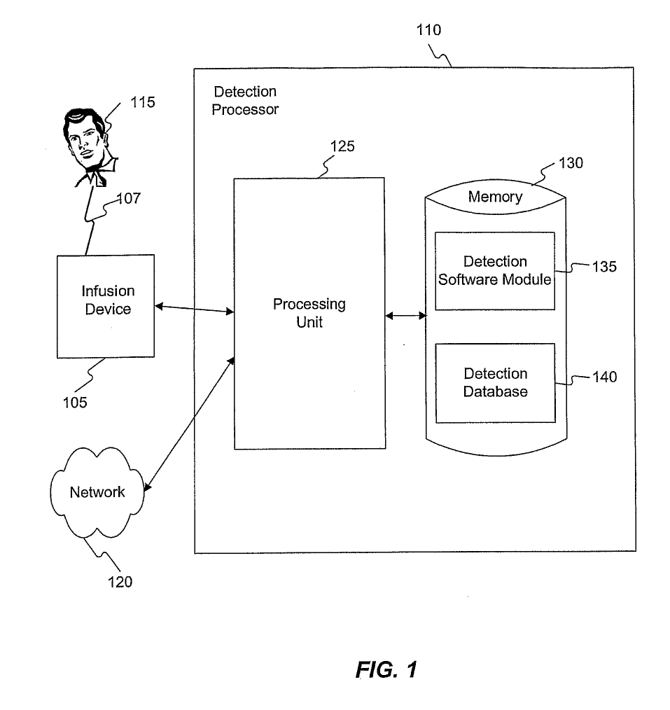

Consistent with an embodiment of the present invention, the aforementioned memory, processing unit, and other components may be implemented in an occlusion detecting system, such as an exemplary occlusion detecting system 100 of FIG. 1. Any suitable combination of hardware, software, and/or firmware may be used to implement the memory, processing unit, or other components. By way of example, the memory, processing unit, or other components may be implemented with a detection processor 110, in combination with system 100. The aforementioned system and processors are exemplary and other systems and processors may comprise the aforementioned memory, processing unit, or other components, consistent with embodiments of the present invention.

Furthermore, the invention may be practiced in an electrical circuit comprising discrete electronic elements, packaged or integrated electronic chips containing logic gates, a circuit utilizing a microprocessor, or on a single chip containing electronic elements or microprocessors. The invention may also be practiced using other technologies capable of performing logical operations such as, for example, AND, OR, and NOT, including but not limited to mechanical, optical, fluidic, and quantum technologies. In addition, the invention may be practiced within a general purpose computer or in any other circuits or systems.

By way of a non-limiting example, FIG. 1 illustrates system 100 in which the features and principles of the present invention may be implemented. As illustrated in the block diagram of FIG. 1, system 100 may include infusion device 105, detection processor 110, a user 115, and a network 120. While processor 110 may be hardwired to device 105, processor 110 may communicate to other devices or processors via network 120. In another embodiment (not shown) processor 110 may not be hardwired to device 105, but may communicate with device 105 over a network similar to network 120.

Device 105, for example, may comprise, but is not limited to, an ambulatory infusion pump. Device 105 may deliver material, such as insulin or other medicines, for example, through a tube and hollow needle (for example, an infusion set 107) into the body of user 115. While device 105 and processor 110 are shown in FIG. 1 in separate blocks they may be constructed in one or in separate packages. User 115 may be a subject, for example, desiring to detect an occlusion using processor 110.

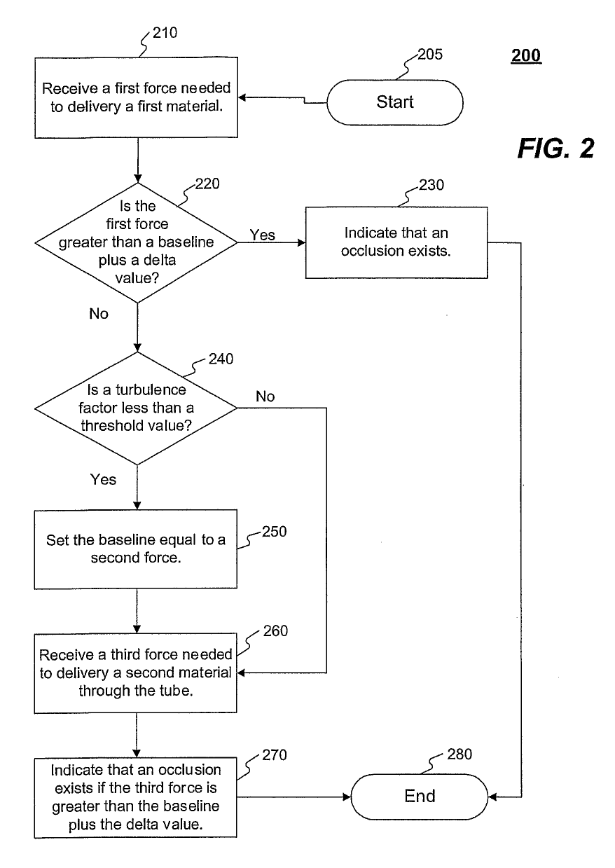

Detection processor 110 may include a processing unit 125 and a memory 130. Memory 130 may include a detection software module 135 and a detection database 140. The software module 135, residing in memory 130, may be executed on processing unit 125, may access database 140, and may implement processes for detecting an occlusion such as, for example, the method described below with respect to FIG. 2. Notwithstanding, processor 110 may execute other software modules and implement other processes different than or in addition to the aforementioned.

While processor 110 may be included in the same package as device 105 as described above, processor 110 may be implemented using a personal computer, network computer, mainframe, or other similar microcomputer-based workstation. Processor 110 may though comprise any type of computer operating environment, such as a hand-held device, a multiprocessor system, a microprocessor-based or programmable sender electronic device, a minicomputer, a mainframe computer, and the like. Processor 110 may also be practiced in distributed computing environments where tasks are performed by remote processing devices. Furthermore, processor 110 may comprise a mobile terminal, such as a smart phone, a cellular telephone, a cellular telephone utilizing wireless application protocol (WAP), personal digital assistant (PDA), intelligent pager, portable computer, a hand held computer, a conventional telephone, or a facsimile machine. The aforementioned systems and devices are exemplary and processor 110 may comprise other systems or devices.

Network 120 may comprise, for example, a local area network (LAN) or a wide area network (WAN). Such networking environments are commonplace in offices, enterprise-wide computer networks, intranets, and the Internet. When a LAN is used as network 120, a network interface located at any of device 105 and processor 110 may be used to interconnect them. When network 120 is implemented in a WAN networking environment, such as the Internet, device 105 or processor 110 may typically include an internal or external modem (not shown) or other means for establishing communications over the WAN. Further, in utilizing network 120, data sent over network 120 may be encrypted to insure data security by using known encryption/decryption techniques.

In addition to utilizing a wire line communications system as network 120, a wireless communications system, or a combination of wire line and wireless may be utilized as network 120 in order to, for example, exchange web pages via the Internet, exchange e-mails via the Internet, or for utilizing other communications channels. Wireless can be defined as radio transmission via the airwaves. However, it may be appreciated that various other communication techniques can be used to provide wireless transmission, including infrared line of sight, cellular, microwave, satellite, packet radio, and spread spectrum radio. The processors in the wireless environment can be any mobile terminal, such as the mobile terminals described above. Wireless data may include, but is not limited to, paging, text messaging, e-mail, Internet access and other specialized data applications specifically excluding or including voice transmission.

System 100 may also transmit data by methods and processes other than, or in combination with, network 120. These methods and processes may include, but are not limited to, transferring data via, diskette, CD ROM, flash memory sticks, facsimile, conventional mail, an interactive voice response system (IVR), or via voice over a publicly switched telephone network.

FIG. 2 is a flow chart setting forth the general stages involved in an exemplary method 200 for detecting an occlusion consistent with the invention. Exemplary ways to implement the stages of method 200 will be described in greater detail below. Exemplary method 200 begins at starting block 205 and proceeds to stage 210 where process 110 may receive a signal corresponding to a first force needed to deliver a first material. For example, in the context of an insulin infusion pump, the first force may be the force needed to deliver an insulin dose through infusion set 107 into the body of user 115.

From stage 210, where processor 110 receives the signal corresponding to the first force needed to delivery the first material, exemplary method 200 may continue to decision block 220 where processor 110 may determine whether the first force is greater than a baseline value plus a delta value. If processor 110 determined at decision block 220 that the first force is greater than the baseline value plus the delta value, exemplary method 200 may continue to stage 230 where processor 110 may indicate that an occlusion exists. If processor 110 determines at decision block 220, however, that the first force is not greater than the baseline value plus the delta value, exemplary method 200 may continue to decision block 240 where processor 110 may determine whether a turbulence factor is less than a threshold value. For example, the turbulence factor may be a low-pass filtered version of the absolute value of the difference between the first force and a second force. The second force may comprise a low-pass filtered version of the first force. If processor 110 determined at decision block 240, that the turbulence factor is less than the threshold value, exemplary method 200 may continue to stage 250 where the processor 110 may set the baseline value equal to the second force.

Once processor 110 sets the baseline value equal to the second force, in stage 250, or from decision block 240 if processor 110 determines that the turbulence factor is not less than the threshold value, exemplary method 200 advances to stage 260 where processor 110 may determine a third force needed to deliver a second material. For example, in the context of an insulin infusion pump, the third force may be the force needed to deliver a next insulin dose through infusion set 107 into the body of user 115.

After processor 110 determines the third force needed to deliver the second material in stage 260, exemplary method 200 may continue to stage 270 where processor 110 may indicate that an occlusion exists if the third force is greater than the baseline value plus the delta value. From stage 270 where processor 110 indicates that an occlusion exists if the third force is greater than the baseline value plus the delta value, or from stage 230 where processor 110 indicates that an occlusion exists, exemplary method 200 may then end at stage 280. Consistent with embodiments of the invention, any or all of the stages of exemplary method 200 may be repeated, for example, to provide multiple doses through infusion set 107 into the body of user 115.

While certain features and embodiments of the invention have been described, other embodiments of the invention will be apparent to those skilled in the art from consideration of the specification and practice of the embodiments of the invention disclosed herein. Furthermore, although embodiments of the present invention have been described as being associated with data stored in memory and other storage mediums, one skilled in the art will appreciate that these aspects can also be stored on or read from other types of computer-readable media, such as secondary storage devices, hard disks, floppy disks, a CD-ROM, a carrier wave from the Internet, or other forms of RAM or ROM. Further, the steps of the disclosed methods may be modified in any manner, including by reordering steps and/or inserting or deleting steps, without departing from the principles of the invention.

It is intended, therefore, that the specification be considered as exemplary only, with a true scope and spirit of the invention being indicated by the following claims and their full scope of equivalents.

What is claimed is:

1. A method for detecting an occlusion, the method comprising:
receiving a signal corresponding to a first force needed to deliver a first material;
indicating that an occlusion exists if the first force is greater than a baseline value plus a delta value;
setting, if the first force is less than or equal to the baseline value plus the delta value, and if a turbulence factor is less than a threshold value, the baseline value equal to a second force;
receiving a signal corresponding to a third force needed to deliver a second material; and
indicating that an occlusion exists if the third force is greater than the baseline value plus the delta value.

2. The method of claim 1, wherein indicating that an occlusion exists if the first force is greater than the baseline value plus the delta value further comprises assigning the baseline value a value equal to the force necessary to deliver the first material in an un-occluded state.

3. The method of claim 1, wherein indicating that an occlusion exists if the first force is greater than the baseline value plus the delta value further comprises assigning the delta value a value selected to create a desired level of sensitivity.

4. The method of claim 1, wherein setting the baseline value equal to the second force further comprises the second force being a low-pass filtered version of the first force.

5. The method of claim 1, wherein setting the baseline value equal to the second force further comprises the turbulence factor being a low-pass filtered version of the absolute value of the difference between the first force and the second force.

6. The method of claim 1, further comprising providing the first material and the second material wherein the first material and the second material comprise at least one of insulin and medicine.

7. The method of claim 1, wherein receiving the signal corresponding to the first force further comprises receiving the signal corresponding to the first force from a device configured to infuse the first material into a subject's body.

8. The method of claim 7, wherein receiving the signal corresponding to the first force from the device further comprises receiving the signal corresponding to the first force from the device comprising an ambulatory infusion pump.

9. The method of claim 1, wherein receiving the signal corresponding to the third force further comprises receiving the signal corresponding to the third force from a device configured to infuse the second material into a subject's body.

10. The method of claim 9, wherein receiving the signal corresponding to the third force from the device further comprises receiving the signal corresponding to the third force from the device comprising an ambulatory infusion pump.

11. The method of claim 1, further comprising setting the delta value as a function of a number of delivered units of material delivered within a period of time.

12. A system for detecting an occlusion, the system comprising:
a memory storage for maintaining a plurality of data registers; and
a processing unit coupled to the memory storage, wherein the processing unit is operative to
receive a signal corresponding to a first force needed to deliver a first material;
indicate that an occlusion exists if the first force is greater than a baseline value plus a delta value;
set, if the first force is less than or equal to the baseline value plus the delta value, and if a turbulence factor is less than a threshold value, the baseline value equal to a second force;
receive a signal corresponding to a third force needed to deliver a second material; and
indicate that an occlusion exists if the third force is greater than the baseline value plus the delta value.

13. The system of claim 12, wherein the processing unit being operative to indicate that an occlusion exists if the first force is greater than the baseline value plus the delta value further comprises the processing unit being operative to assign the baseline value a value equal to the force necessary to deliver the first material in an un-occluded state.

14. The system of claim 12, wherein the processing unit being operative to indicate that an occlusion exists if the first force is greater than the baseline value plus the delta value further comprises the processing unit being operative to assign the delta value a value selected to create a desired level of sensitivity.

15. The system of claim 12, wherein the processing unit being operative to set the baseline value equal to the second force further comprises the second force being a low-pass filtered version of the first force.

16. The system of claim 12, wherein the processing unit being operative to set the baseline value equal to the second force further comprises the turbulence factor being a low-pass filtered version of the absolute value of the difference between the first force and the second force.

17. The system of claim 12, further comprises the processing unit being operative to provide the first material and the second material wherein the first material and the second material comprise at least one of insulin and medicine.

18. The system of claim 12, wherein the processing unit being operative to receive the signal corresponding to the first force further comprises the processing unit being operative to receive the signal corresponding to the first force from a device configured to infuse the first material into a subject's body.

19. The system of claim 18, wherein the processing unit being operative to receive the signal corresponding to the first force from the device further comprises the processing unit being operative to receive the signal corresponding to the first force from the device comprising an ambulatory infusion pump.

20. The system of claim 12, wherein the processing unit being operative to receive the signal corresponding to the third force further comprises the processing unit being operative to receive the signal corresponding to the third force from a device configured to infuse the second material into a subject's body.

21. The system of claim 20, wherein the processing unit being operative to receive the signal corresponding to the third force from the device further comprises the processing unit being operative to receive the signal corresponding to the third force from the device comprising an ambulatory infusion pump.

22. The system of claim 12, wherein the processing unit is further configured to set the delta value as a function of a number of delivered units of material delivered within a period of time.

23. A computer-readable medium which stores a set of instructions which when executed performs a method for detecting an occlusion, the method executed by the set of instructions comprising:
receiving a signal corresponding to a first force needed to deliver a first material;
indicating that an occlusion exists if the first force is greater than a baseline value plus a delta value;
setting, if the first force is less than or equal to the baseline value plus the delta value, and if a turbulence factor is less than a threshold value, the baseline value equal to a second force;
receiving a signal corresponding to a third force needed to deliver a second material; and
indicating that an occlusion exists if the third force is greater than the baseline value plus the delta value.

24. The computer-readable medium of claim 23, wherein indicating that an occlusion exists if the first force is greater than the baseline value plus the delta value further comprises assigning the baseline value a value equal to the force necessary to deliver the first material in an un-occluded state.

25. The computer-readable medium of claim 23, wherein indicating that an occlusion exists if the first force is greater than the baseline value plus the delta value further comprises assigning the delta value a value selected to create a desired level of sensitivity.

26. The computer-readable medium of claim 23, wherein setting the baseline value equal to the second force further comprises the second force being a low-pass filtered version of the first force.

27. The computer-readable medium of claim 23, wherein setting the baseline value equal to the second force further comprises the turbulence factor being a low-pass filtered version of the absolute value of the difference between the first force and the second force.

28. The computer-readable medium of claim 23, further comprising providing the first material and the second material wherein the first material and the second material comprise at least one of insulin and medicine.

29. The computer-readable medium of claim 23, wherein receiving the signal corresponding to the first force further comprises receiving the signal corresponding to the first force from a device configured to infuse the first material into a subject's body.

30. The computer-readable medium of claim 29, wherein receiving the signal corresponding to the first force from the device further comprises receiving the signal corresponding to the first force from the device comprising an ambulatory infusion pump.

31. The computer-readable medium of claim 23, wherein receiving the signal corresponding to the third force further comprises receiving the signal corresponding to the third force from a device configured to infuse the second material into a subject's body.

32. The computer-readable medium of claim 31, wherein receiving the signal corresponding to the third force from the device further comprises receiving the signal corresponding to the third force from the device comprising an ambulatory infusion pump.

33. The computer-readable medium of claim 23, further comprising setting the delta value as a function of a number of delivered units of material delivered within a period of time.
